(19) United States
(12) Patent Application Publication
(10) Pub. No.: US 2003/0139367 A1
(43) Pub. Date: Jul. 24, 2003

(54) Gene therapy composition for treating viral myocarditis

(76) Inventors: Eun-Seok Jeon, Seoul (KR); Byung-Kwan Lim, Seoul (KR); Jong-Mook Kim, Seoul (KR); Sunyoung Kim, Seoul (KR)

Correspondence Address: David A. Einhorn, Esq., Anderson Kill & Olick, P.C., 1251 Avenue of the Americas, New York, NY, 10020, US

(21) Appl. No.: 10/348,483

(22) Filed: Jan. 17, 2003

Related U.S. Application Data
(60) Provisional application No. 60/350,049, filed on Jan. 18, 2002.

(51) Int. Cl. A61K 48/00

(52) U.S. Cl. 514/44

(57) ABSTRACT

An intracardiac injection composition for treating viral myocarditis in a mammal, which comprises a DNA encoding interleukin-1 receptor antagonist (IL-1Ra) and a pharmaceutically acceptable carrier

### FIELD OF THE INVENTION

The present invention relates to an intracardiac injection composition for treating viral myocarditis, which comprises a DNA encoding interleukin-1 receptor antagonist (IL-1Ra); and a method for treating viral myocarditis by injecting same into the myocardium of a subject.

### BACKGROUND OF THE INVENTION

Among the etiological viruses of viral myocarditis, enteroviruses, in particular coxsackievirus B, are the most common (Feldman A. and McNamara D., N Engl J Med, 343:1388-1398(2000); I. -W. Seong, et al., N Engl J Med, 345:379(2001); Woodruff J. F., Am J Pathol, 101:425-483(1980); and Li Y. et al., Circulation, 101:231-234(2000)).

In viral myocarditis, virus proliferation in myocytes can induce direct cytotoxicity, independent of an immune response, and some coxsackieviral proteins can cause direct myocyte damage (Badorff C. et al., Nat Med, 5:320-326(1999)). The immune responses may induce tissue damage by: 1) the protective host response to remove virus-infected myocytes, and/or, 2) inappropriate cardiac injury caused by sensitized T-lymphocytes (Huber S. A. and Pfaeffle B., J Virol, 68:5126-5132(1994)). This immune response should be specific, attacking only infected cells, but such an imbalance in the immune response may lead to either an overwhelming virus-induced myocardial injury or predominantly immune-mediated tissue damage (Knowlton K. U. and Badorff C., Circ Res, 85:559-561(1999)).

IL-1Ra inhibits many actions of IL-1 by competing for its receptor. The balance between endogenous IL-1 and IL-1Ra in vivo is an important determinant of the host response to the infection (Arend W. P. et al., Annu Rev Immunol, 16:24-55(1998)). In a murine myocarditis model, a increase in the IL-1β level has been found to correlates with the myocardial fibrosis (Shioi T. et al., Circulation, 94:2930-2937(1996)), while a increase in the serum IL-1Ra level, by continuous IL-1Ra infusion, improves the survival rates and decreases myocardial inflammation and fibrosis (Rose N. and Hill S., Clin Immunol Immunopathol, 83:S92-S99(1996)).

IL-1β has negative inotropic effects and cytotoxicity through excessive NO production by iNOS in viral myocarditis (Nakano A. et al., supra; and Mikami S. et al., Circ Res, 81:504-511(1997)). IL-1β may also activate fibroblasts, which affect the remodelling process after myocardial injury (Bouluyt M. C. et al., Circ Res, 75:25-32(1994)). To inhibit the IL-1 response in the cells that express IL-1 receptor, a 10- to 100-fold excess of IL-1Ra is required (Granowitz E. V. et al., Lancet, 338:1423-1424(1991)).

Local expression of IL-1Ra may have more therapeutic effect on viral myocarditis than systemic infusion, because of the short half-life of IL-1Ra in serum.

The present inventors have endeavored to develop a novel delivery system of IL-1Ra for treating viral myocarditis in a mammal, and, as a result, have discovered that myocardiac injection of a expression vector containing a DNA encoding IL-1Ra is an excellent method for treating viral myocarditis, the local expression of IL-1Ra suppressing viral replication, myocardial inflammation, and subsequent fibrosis in the heart, and improving the survival rates.

### SUMMARY OF THE INVENTION

Accordingly, it is an object of the present invention to provide a pharmaceutical composition for treating viral myocarditis in a mammal.

It is another object of the present invention to provide a method for treating viral myocarditis in a mammal.

In accordance with one aspect of the present invention, there is provided an intracardiac injection composition for treating viral myocarditis in mammal, which comprises a DNA encoding interleukin-1 receptor antagonist and pharmaceutically acceptable carriers.

In accordance with another aspect of the present invention, there is provided a method for treating viral myocarditis in a mammal, which comprises administering an effective amount of the DNA encoding interleukin-1 receptor antagonist thereto via intracardiac injection.

### BRIEF DESCRIPTION OF THE DRAWINGS

The above and other objects and features of the present invention will become apparent from the following description of the invention, when taken in conjunction with the accompanying drawings, in which:

FIG. 1 displays the structure of the inventive expression vector pCK-IL-1 Ra (Hatched box: major immediate early promoter of human cytomegalovirus; dotted box: exon; wavy line: intron; pA: poly A tract; Kan: kanamycin resistance gene; and ColEI: E. coli origin of replication).

FIG. 2 shows the time-dependent change in the heart hIL-1Ra level in a mouse, after intracardiac injection of pCK-IL-1Ra;

FIG. 3 presents a cumulative survival rate of mice injected with expression vector pCK or pCK-IL-1Ra, after CVB3 infection;

FIG. 4 represents the changes in virus titers in the heart in mice injected with expression vector pCK or pCK-IL-1Ra on days 3, 7 and 14 after CVB3 infection; and

FIG. 5 shows the effects of overexpressed hIL-1Ra on viral myocarditis and fibrosis: myocardial inflammation on day 7 (A, D) and day 14 (B, E), and fibrosis and ventricular dilatation (C, F) on day 14(A to D, H&E staining, magnification ×100; and E and F, Masson's Trichrome staining, magnification ×10).

### DETAILED DESCRIPTION OF THE INVENTION

In the inventive IL-1Ra (IL-1 receptor antagonist) gene therapy, viral myocarditis, e.g., coxsackieviral myocarditis, can effectively be treated by administering the DNA encoding IL-1Ra in the form of an expression vector containing a DNA encoding IL-1Ra via intracardiac injection.

The DNA encoding IL-1Ra of the present invention can be obtained from human peripheral blood lymphocytes or synthesized using a conventional DNA synthesis method. Further, the DNA thus prepared may be inserted to a vector for intracardiac gene therapy, to obtain an expression vector.

The vector for intracardiac gene therapy that may be advantageously used in the present invention is pCK plasmid, which gives a high-level gene expression in a mammal (Lee Y, et al., Biochem. Biophys. Res. Commun., 272:230-5(2000)). pCK contains not only the full length major immediate-early (IE) promoter of human cytomegalovirus (HCMV) but also its entire 5′-untranslated region consisting of the entire exon 1 and intron 1, as well as a part of the exon 2. Further, pCK is designed in such a way that the start codon of the inserted gene coincides with the ATG codon of the original IE gene of HCMV, a feature not found in many other HCMV promoter-based expression vectors. The inventive expression vector obtained by cloning the human IL-1Ra cDNA into plasmid pCK is hereinafter referred to as “pCK-IL-1Ra”.

The present invention includes within its scope an intracardiac injection composition for treating viral myocarditis comprising the DNA encoding IL-1Ra in the form of expression vector containing a DNA encoding IL-1Ra, preferably pCK-IL-1Ra, in association with pharmaceutically acceptable carriers, excipients or other additives, if necessary.

Examples of suitable carriers, excipients, and diluents are lactose, dextrose, sucrose, sorbitol, mannitol, starches, gum acacia, alginates, gelatin, calcium phosphate, calcium silicate, cellulose, methyl cellulose, microcrystalline cellulose, polyvinylpyrrolidone, water, methylhydroxybenzoates, propylhydroxybenzoates, talc, magnesium stearate and mineral oil. The compositions may additionally include lubricating agents, wetting agents, flavoring agents, emulsifiers, preservatives and the like.

The compositions of the present invention may be formulated so as to provide quick, sustained or delayed release of the active ingredient after administration to a patient by employing any of the procedures well known in the art.

The present invention also includes within its scope a method for treating viral myocarditis in a subject by way of administering a therapeutically effective amount of the DNA encoding IL-1Ra in the form of an expression vector containing a DNA encoding IL-1Ra, preferably pCK-IL-1Ra via intracardiac injection.

The intracardiac direct injection of the DNA encoding IL-1Ra works to generate a remarkable synergistic treatment effect, which can be sustained for more than 14 days, such a synergistic effect cannot be achieved by any other mode of administration. The expression of a high level of IL-1Ra in the heart suppresses viral replication, myocardial inflammation, and subsequent fibrosis in the heart, and improves the survival rates of the subjects.

The effective amount of the DNA encoding IL-1Ra as an active ingredient may range from about 0.05 to 500 mg/kg, preferably 0.5 to 50 mg/kg body weight, and can be administered in a single dose or in divided doses. However, it should be understood that the amount of the active ingredient actually administered ought to be determined in light of various relevant factors including the condition to be treated, the age and weight of the individual patient, and the severity of the patient's symptom; and, therefore, the above dose should not be intended to limit the scope of the invention in any way.

The following Examples are intended to further illustrate the present invention without limiting its scope.

Further, percentages given below for solid in solid mixture, liquid in liquid, and solid in liquid are on a wt/wt, vol/vol and wt/vol basis, respectively, and all the reactions were carried out at room temperature, unless specifically indicated otherwise.

### REFERENCE EXAMPLE

### Statistics

Data were presented as mean±SEM. Survival rate was analyzed using the Kaplan-Meier method. The student's t-test was used for the analysis of numeric parameters (SPSS 10.0 for Windows, SPSS Inc.). Differences were considered significant at p <0.05.

### Example 1

Cloning of hIL-1Ra and Construction of Expression Vector

cDNA encoding hIL-1Ra was cloned from total RNA prepared from human peripheral blood lymphocytes by reverse transcription(RT)-polymerase chain reaction (PCR). PCR primers were: 5′-AAGCTTATGGAAATCTGCAGAGGCCTCCGCAGTCAC-3′ (SEQ ID NO:1) and 5′-GTCGACCTACTCGTCCTCCTGGAAGTAGAATTTGGT-3′ (SEQ ID NO:2).

The amplified cDNA was initially cloned into the PCR product cloning site of pGEM-72f(+) plasmid (Promega, Wis., USA). Following sequence confirmation, the hIL-1Ra cDNA was cloned into the HindIII site of the mammalian expression vector pCK (Lee Y, et al., Biochem. Biophys. Res. Commun., 272:230-5(2000)) to obtain plasmid pCK-IL-1Ra, which was purified using an EndoFree plasmid Maxi prep kit (Qiagen,Valencia, Calif., USA), diluted in phosphate-buffered saline (pH 7.4) to 1 μg/μl and stored at −20° C. before use.

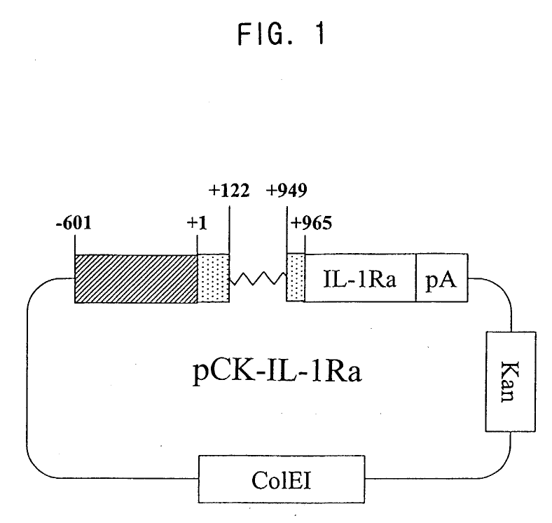

FIG. 1 displays the structure of the inventive expression vector pCK-IL-1Ra (Hatched box: major immediate early promoter of human cytomegalovirus; dotted box: exon; wavy line: intron; pA: poly A tract; Kan: kanamycin resistance gene; and ColEI: E. coli origin of replication).

### Example 2

Establishment of the Coxsackieviral Myocarditis Models

(Step 1) Intracardiac Injection of Plasmid cDNA

All procedures were reviewed and approved by the Animal Care and Use Committee at Soon Chun Hyang University Hospital in Bucheon, Korea.

Sixty 8-week-old inbred female Balb/C mice were anesthetized by intraperitoneal injection of a mixture of Ketamine (100 mg/kg) and Xylazine (5 mg/kg). After a vertical neck incision and blunt dissection, a tracheostomy was performed. The mice were mechanically ventilated on an animal ventilator (CWE Inc.) at a tidal volume of 0.5 cm³/stroke and a respiratory rate of 100 strokes/minute. Left thoracotomy was performed at the fifth intercostal space, and after the pericardium was opened, the apex of the heart was injected with 40 μg/40 μl of the cDNA of pCK(n=15) or pCK-IL-1Ra(n=45) using an insulin syringe. Successful injection into the myocardium produced an obvious blanching in the apex. The skin at the neck and chest was closed and the mice were extubated when spontaneous movement was observed. Immediate post-operative mortality was less than 5%, primarily a result of pneumothorax. The day of plasmid vector injection was defined as day −3.

(Step 2) Induction of Coxsackieviral Myocarditis

Coxsackievirus B3(CVB3) was derived from the infectious cDNA copy of the cardiotropic H3 strain of CVB3 (H3 variant of the Woodruff strain) (Knowlton K. U. et al., J Virol, 70:7811-7818(1996)). Viruses were titered using the plaque-forming assay on HeLa cells.

Three days after the plasmid injection (day 0), mice of the pCK-injected control group (n=15) and 35 out of 45 mice of pCK-IL-1Ra-injected group were infected by intraperitoneal injection with 10⁴ PFU of CVB3. The above two groups were designated pCK-CVB3 and IL-1Ra-CVB3, respectively. Mice were euthanized and serum, hearts, and livers were collected at days 3, 5, 7, 9 and 14.

### Example 3

Treatment Effect of pCK-IL-1Ra on the Coxsackieviral Myocarditis

(1) Time-dependent Changes in the in Vivo Expression Level of IL-1Ra

To investigate the changes in the expression level of human IL-1Ra by delivered pCK-IL-1Ra in the heart and serum, IL-1Ra expressed in the heart and serum was measured using commercially available ELISA kit for hIL-1Ra (R&D Systems, Minneapolis, Minn., USA), according to the manufacturer's recommendations.

Specifically, the apical part of the heart from the mice treated as described in (Step 1) of Example 2 was homogenized in a lysis buffer (25 mM Tris-HCl pH 7.4, 50 mM NaCl, 0.5 % Na-deoxycholate, 2% NP-40, 0.2% sodium dodecyl sulfate, and protease inhibitors). After centrifugation, the resulting supernatant was used to measure the level of hIL-1Ra (pg/ml), which was normalized to the amount (mg) of total protein extracted from the heart. The serum was directly used for hIL-1Ra assay. Serial dilutions of recombinant murine IL-1Ra were used as a standard.

The levels of heart hIL-1Ra measured on day 3 (2160±1651 pg/ml/mg of total protein), day 5 (2234±713), day 7 (695±394), day 9 (1062±383) and day 14 (1221±456), were significantly higher than the corresponding values for the control (24±3).

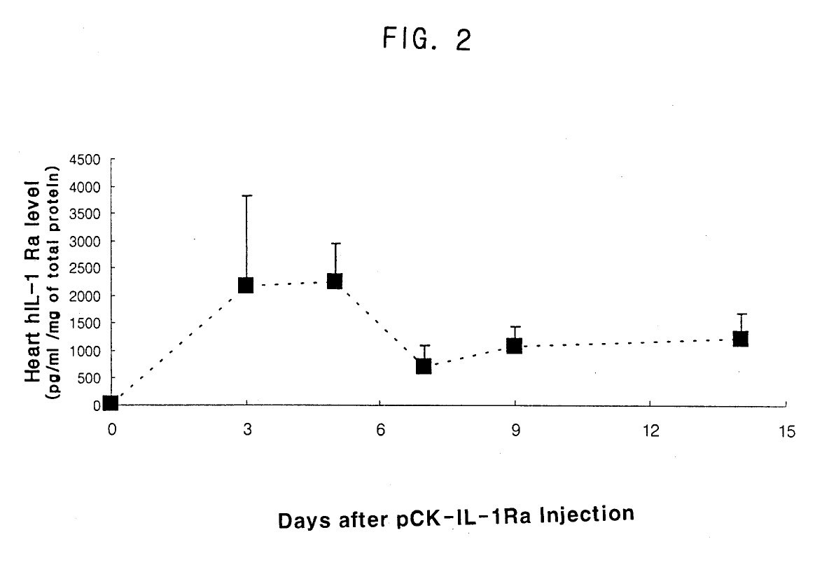

FIG. 2 shows the time-dependent change in the in vivo expression level of human IL-1Ra in the heart, after the intracardiac injection of pCK-IL-1Ra. As shown in FIG. 2, the level of hIL-1Ra in the heart was highest on day 5, and this level was sustained for two weeks. In more than 70 % of pCK-IL-1Ra mice, the expressed hIL-1Ra level was at least three-fold higher than the control. Serum hIL-1Ra was not significantly elevated.

(2) Survival Rates

None of the ten pCK-IL-1Ra-injected mice without CVB3 infection died during four weeks after the operation.

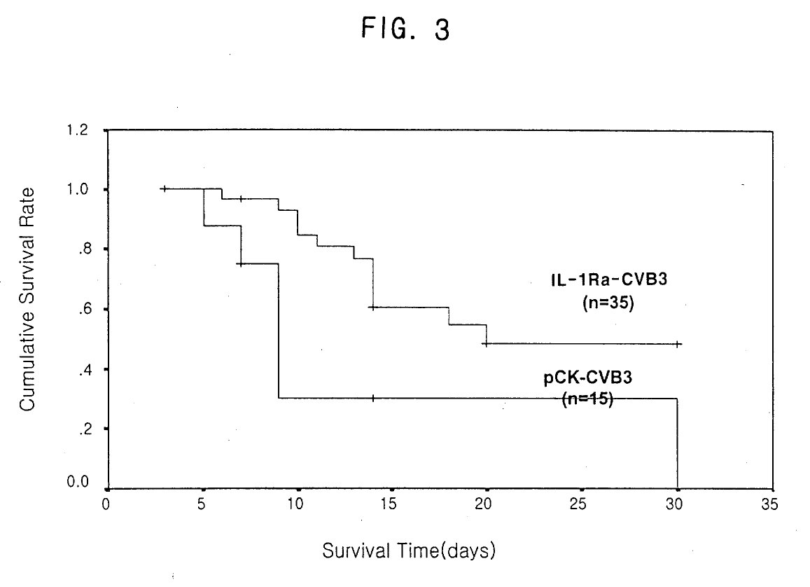

The pCK-IL-1Ra-injected mice with CVB3 infection (IL-1Ra-CVB3, n=35) showed a significantly higher survival rate(77%) than the pCK-CVB3 group (n=15, 30%, p<0.01) at day 14. At the end of four weeks after the infection, the survival rate of IL-1Ra-CVB3 was significantly higher than that of pCK-CVB3 (48% versus 10%, p<0.01, FIG. 3).

These results suggest that expressed hIL-1Ra had a therapeutic effect on the mortality of viral myocarditis.

(3) Changes of Viral Titers in the Hearts

The base parts of the hearts were homogenized in DMEM (Biowhittaker, US) containing 2% fetal calf serum (FCS). Cellular debris was removed by centrifugation, and viral titers in the supernatant were determined by the plaque forming assay (Knowlton K. U. et al., J Virol, 70:7811-7818(1996)).

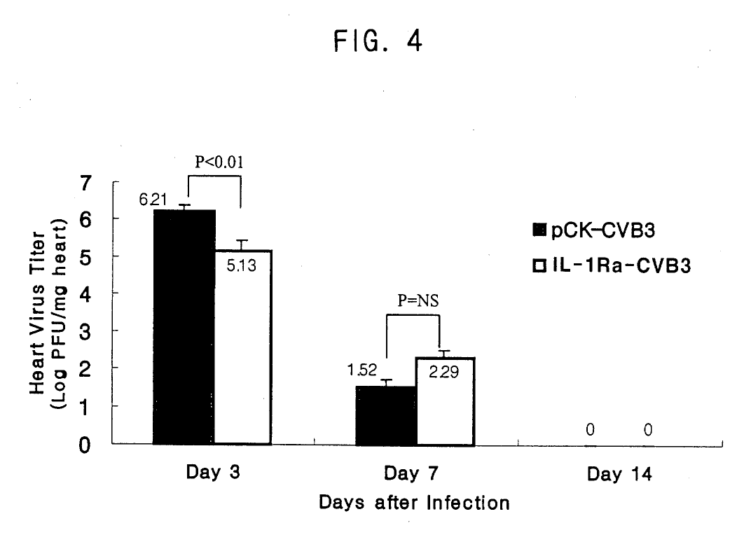

As can be seen from FIG. 4, the viral titers in the hearts of IL-1Ra-CVB3 mice were significantly lower than those of pCK-CVB3 mice at day 3 (5.13±0.19 versus 6.21±0.16 log₁₀ PFU/mg heart, p=0.002). On day 7, the viral titers decreased in both groups, showing no significant differences (1.52±0.30 versus 2.29±0.23, p>0.05, FIG. 4).

(4) Changes of Inflammation and Fibrosis in the Hearts

The apical parts of the hearts were fixed in 10% formalin, embedded in paraffin, and stained with hematoxylin-eosin (H&E) and Masson's trichrome. Sections were analysed “blind” and graded for cardiac inflammation by two histologists. The sections were scored arbitrarily as 0 when normal, and 1 to 4 according the degree of myocarditis in view of spread and confluence of inflammation.

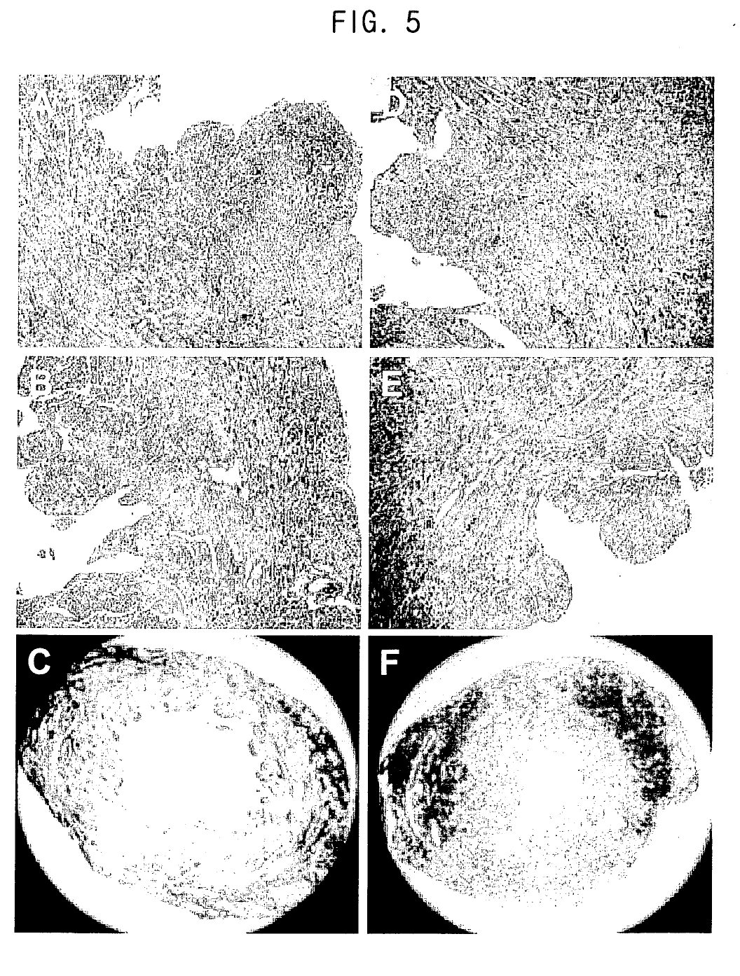

The IL-1Ra-CVB3 group exhibited markedly decreased myocardial inflammation on days 7 (3.0±0.5) and 14 (2.0±0.5) when compared with the pCK-CVB3 group (2.0±0.5 at day 7 and 1.5±0.5 at day 14, p<0.05; FIG. 5A to D, H&E staining). The fibrosis on day 14 was also much less pronounced for the IL-1Ra-CVB3 group (FIG. 5 E and F, Masson's trichrome staining).

While the invention has been described with respect to the above specific embodiments, it should be recognized that various modifications and changes may be made to the invention by those skilled in the art which also fall within the scope of the invention as defined by the appended claims.

What is claimed is:

1. An intracardiac injection composition for treating viral myocarditis in a mammal, which comprises a DNA encoding interleukin-1 receptor antagonist (IL-1Ra) and a pharmaceutically acceptable carrier.

2. The composition of claim 1, wherein the DNA encoding IL-1Ra is contained in an expression vector.

3. The composition of claim 2, wherein the expression vector is pCK-IL-1Ra.

4. The composition of claim 1, wherein the mammal is human.

5. A method for treating viral myocarditis in a mammal, which comprises administering thereto an effective amount of a DNA encoding IL-1Ra via intracardiac injection.

6. The method of claim 5, wherein the DNA encoding IL-1Ra is contained in an expression vector.

7. The method of claim 6, wherein the expression vector is pCK-IL-1Ra.

8. The method of claim 5, wherein the mammal is human.

9. The method of claim 5, wherein the effective amount of the DNA encoding IR-1Ra ranges from 0.05 to 500 mg/kg body weight.


SEQUENCE LISTING (2 sequences)

SEQ ID NO: 1 - DNA, 36 bp, Artificial Sequence
  primer
aagcttatggaaatctgcagaggcctccgcagtcac

SEQ ID NO: 2 - DNA, 36 bp, Artificial Sequence
  primer
gtcgacctactcgtcctcctggaagtagaatttggt